(19) United States
(12) Patent Application Publication
(10) Pub. No.: US 2004/0175352 A1
(43) Pub. Date: Sep. 9, 2004

(54) Microbicidal compositions including an ionene polymer and 2,4,4-trichloro-2-hydroxydiphenyl ether, and methods of using the same

(76) Inventors: David Oppong, Cordova, TN (US); Graciella H. Vunk, Olive Branch, MS (US); Russell E. Fues, Memphis, TN (US)

Correspondence Address: KILYK & BOWERSOX, P.L.L.C., 53 A EAST LEE STREET, WARRENTON, VA, 20186, US

(21) Appl. No.: 10/383,121

(22) Filed: Mar. 6, 2003

(51) Int. Cl. A61K 31/765

(52) U.S. Cl. 424/78.17

(57) ABSTRACT

Microbicidal compositions including (a) an ionene polymer and (b) 2,4,4′-trichloro-2′-hydroxydiphenyl ether are described. The ionene polymer and 2,4,4′-trichloro-2′-hydroxydiphenyl ether can be present in a synergistically effective amount to control the growth of at least one microorganism. Methods for controlling the growth of microorganisms with the compositions are also disclosed.

### BACKGROUND

Many industrial materials and media when wet or subjected to treatment in water are susceptible to bacterial, fungal, and/or algal deterioration or degradation. These industrial materials and media include, but are not limited to, for example, wood pulp, wood chips, lumber, adhesives, coatings, animal hides, paper mill liquors, pharmaceutical formulations, cosmetic formulations, toiletry formulations, geological drilling lubricants, petrochemicals, agrochemical compositions, paints, leathers, wood, metalworking fluids, cooling water, recreational water, influent plant water, waste water, pasteurizers, retort cookers, tanning liquors or solutions, starch, proteinaceous materials, acrylic latex paint emulsions, and textiles.

To control deterioration or degradation caused by microorganisms, various industrial microbicides are used. Workers in the trade have continued to seek improved biocides that have low toxicity, are cost effective, and are also capable of exhibiting a prolonged biocidal effect against a wide variety of microorganisms with regular use.

Ionene polymers, i.e., cationic polymers containing quarternary nitrogens in the polymer backbone, are one group of biocides used in controlling bacteria and algae in various aqueous systems. Ionene polymers have a variety of uses in aqueous systems such as, for example, microbicides, bactericides, and algicides, as well as controlling and even preventing biofilm and slime formation. Illustrative examples of these polymers, their uses and preparation are described in U.S. Pat. Nos. 3,874,870; 3,898,336; 3,931,319; 4,027,020; 4,054,542; 4,089,977; 4,111,679; 4,506,081; 4,581,058; 4,778,813; 4,970,211; 5,051,124; and 5,093,078, the disclosures of all of which are incorporated in their entireties by reference herein. While generally effective as algicides and bactericides, most ionene polymers are less effective against many groups of fungi.

The use of halogenated diphenyl ether, including triclosan, to control microorganisms has also been described in, for example, U.S. Pat. No. 3,629,477 and GB 1,592,011, both of which are incorporated in their entirety herein by reference. Although, generally acceptable as a microbicide, triclosan tends to be ineffective against certain microbes at relatively low concentrations. For instance, low concentrations of triclosan tend to be ineffective against certain fungi and some common troublesome industrial bacteria such as Pseudomonas sp. Systems or products requiring high concentrations of triclosan tend to be expensive or uneconomical, and limit its use in a number of applications.

Accordingly, there is a need in the art for a microbicide that overcomes these and other problems.

### SUMMARY

It is a feature of this invention to provide a microbicidal composition capable of controlling the growth of at least one microorganism, for example, fungi, bacteria, algae, and mixtures thereof, over prolonged periods of time. It is an additional feature of this invention to provide such compositions which are economical to use. Methods of controlling the growth of at least one microorganism are also features of this invention.

Compositions and processes useful for controlling the growth of one or more microorganisms are described. Compositions and methods for preventing damage during storage caused by microorganisms, such as bacteria, fungi, algae, and mixtures thereof, are described. The compositions include an ionene polymer and a halogenated diphenyl ether, 2,4,4-trichloro-2-hydroxydiphenylether, also known as triclosan.

The present invention provides a composition comprising an ionene polymer and triclosan, where the components are present in a combined amount synergistically effective to control the growth of at least one microorganism.

The present invention provides a method for controlling the growth of at least one microorganism in or on a product, material, or medium susceptible to attack by the microorganism. This method includes the step of adding to the product, material, or medium a composition of the present invention in an amount synergistically effective to control the growth of the microorganism. The synergistically effective amount varies in accordance with the product, material, or medium to be treated and can, for a particular application, be routinely determined by one skilled in the art in view of the disclosure provided herein.

The present invention also embodies the separate addition of an ionene polymer and triclosan to products, materials, or media. According to this embodiment, the components are individually added to the products, materials, or media so that the final amount of each component present at the time of use is that amount synergistically effective to control the growth of at least one microorganism.

The compositions of the present invention are useful in preserving or controlling the growth of at least one microorganism in various types of industrial products, media, or materials susceptible to attack by microorganisms. Such media or materials include, but are not limited to, for example, dyes, pastes, lumber, leathers, textiles, pulp, wood chips, tanning liquor, paper mill liquor, polymer emulsions, paints, paper and other coating and sizing agents, metalworking fluids, geological drilling lubricants, petrochemicals, cooling water systems, recreational water, influent plant water, waste water, pasteurizers, retort cookers, pharmaceutical formulations, cosmetic formulations, and toiletry formulations.

The composition can also be useful in agrochemical formulations for the purpose of protecting seeds or crops against microbial spoilage.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

It is understood that both the foregoing general description and the following detailed description are exemplary and explanatory only, and are not restrictive of the present invention as claimed.

### DETAILED DESCRIPTION OF CERTAIN EMBODIMENTS

The present invention provides a composition to control the growth of at least one microorganism comprising a mixture of an ionene polymer and triclosan, wherein the components are present in a combined amount synergistically effective to control the growth of at least one microorganism. The composition preferably provides superior microbicidal activity at low concentrations against a wide range of microorganisms.

The compositions of the present invention can be used in a method for controlling the growth of at least one microorganism in or on a product, material, or medium susceptible to attack by the microorganism. This method includes the step of adding to the product, material, or medium a composition of the present invention, where the components of the composition are present in synergistically effective amounts to control the growth of the microorganism.

The synergistically effective amount varies in accordance with the material or medium to be treated and can, for a particular application, be routinely determined by one skilled in the art in view of this disclosure.

In lieu of adding the composition of the present invention to a material or medium to be treated, the ionene polymer and a halogenated diphenyl ether, for example, trioclosan, can be separately added to the material or medium to be treated. These components are individually added so that the final amount of the mixture of ionene polymer and triclosan at the time of use is that amount synergistically effective to control the growth of at least one microorganism.

As stated earlier, the compositions of the present invention are useful in preserving various type of industrial products, media, or materials susceptible to attack by at least one microorganism. The compositions of the present invention are also useful in agrochemical formulations for the purpose of protecting seeds or crops against microbial spoilage. These methods of preserving and protecting are accomplished by adding the composition of the present invention to the products, media, or materials in an amount synergistically effective to preserve the products, media, or materials from attack by at least one microorganism or to effectively protect the seeds or crops against microbial spoilage.

According to the methods of the present invention, controlling or inhibiting the growth of at least one microorganism includes the reduction and/or the prevention of such growth.

It is to be further understood that by “controlling” (i.e., preventing) the growth of at least one of microorganism, the growth of the microorganism is inhibited. In other words, there is no growth or essentially no growth of the microorganism. “Controlling” the growth of at least one microorganism maintains the microorganism population at a desired level, reduces the population to a desired level (even to undetectable limits, e.g., zero population), and/or inhibits the growth of the microorganism. Thus, in one embodiment of the present invention, the products, material, or media susceptible to attack by the at least one microorganism are preserved from this attack and the resulting spoilage and other detrimental effects caused by the microorganism. Further, it is also to be understood that “controlling” the growth of at least one microorganism also includes biostatically reducing and/or maintaining a low level of at least one microorganism such that the attack by the microorganism and any resulting spoilage or other detrimental effects are mitigated, i.e., the microorganism growth rate or microorganism attack rate is slowed down and/or eliminated.

When two chemical microbicides are mixed and added to the product, or added separately, three results are possible:

1) The chemicals in the product would produce an additive (neutral) effect.

2) The chemicals in the product would produce an antagonistic effect, or

3) The chemicals in the product would produce a synergistic effect.

An additive effect has no economic advantage over the individual components. The antagonistic effect would produce a negative impact. Only a synergistic effect, which is less likely than either an additive or antagonistic effect, would produce a positive effect and therefore possess economic advantages.

It is known in the microbicidal literature that there is no theoretical method to anticipate additive, antagonistic, or synergistic effects when two biocides are mixed to yield a new formulation. Nor is there a method to predict the relative proportions of the different biocides required to produce one of the three effects described above.

The inventive microbicidal compositions combining an ionene polymer and a halogenated diphenyl ether, for example, triclosan, demonstrate unexpected synergistic effects compared to the respective components alone. Thus, these compositions achieve superior, i.e. greater than additive, microbicidal activity, even at low concentrations, against a wide variety of microorganisms. Examples of these microorganisms include fungi, bacteria, algae, and mixtures thereof, such as, but not limited to, for example, Trichoderma viride, Aspergillus niger, Pseudomonas aeruginosa, Klebsiella pneumoniae, and Chlorella sp. The compositions of the present invention have a low toxicity.

Ionene polymers may be classified according to the repeating unit found in the polymer. This repeating unit results from reactants used to make the ionene polymer.

A first type of ionene polymer that can be used in the present invention comprises the repeating unit of formula (I):

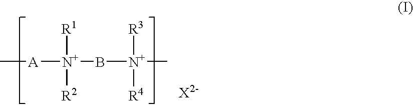

wherein R¹, R², R³, and R⁴ can be identical or different, and are selected from hydrogen, C₁-C₂₀ alkyl, or benzyl. Each C₁-C₂₀ alkyl can be unsubstituted or substituted, for instance, optionally substituted with at least one hydroxyl group. Each benzyl can also be unsubstituted or substituted, for instance, optionally substituted on the benzene moiety with at least one C₁-C₂₀ alkyl group. Preferably, R¹, R², R³, and R⁴ are all methyl or ethyl.

The group “A” is a divalent radical selected from C₁-C₁₀ alkyl, C₂-C₁₀ alkenyl C₂-C₁₀ alkynyl, C₁-C₁₀ hydroxyalkyl, symmetric or asymmetric di-C₁-C₁₀-alkylether, aryl, aryl-C₁-C₁₀-alkyl, or C₁-C₁₀-alkylaryl-C₁-C10-alkyl. The group “A” can be unsubstituted or substituted. Preferably, “A” is C₁-C₅ alkyl, C₂-C₅ alkenyl, C₂-C₅ hydroxyalkyl, or symmetric di-C₂-C₅-alkylether. Most preferably “A” is propylene, 2-hydroxypropylene, or diethylene ether.

The group “B” is a divalent radical, which can be the same as the group “A,” and is selected from C₁-C₁₀ alkyl, C₂-C₁₀ alkenyl, C₂-C₁₀ alkynyl, C₁-C₁₀ hydroxyalkyl, symmetric or asymmetric di-C₁-C₁₀-alkylether, aryl, aryl-C₁-C₁₀-alkyl, or C₁-C₁₀-alkylaryl-C₁-C₁₀-alkyl. The group “B” can be unsubstituted or substituted. Preferably, “B” is C₁-C₅ alkyl, C₂-C₅ alkenyl, C₂-C₅ hydroxyalkyl, aryl, aryl-C₁-C₅-alkyl, or C₁-C₅-alkylaryl-C₁-C₅-alkyl. Most preferably “B” is ethylene, propylene, butylene, or hexamethylene.

The counter ion, X²⁻, is a divalent counter ion, two monovalent counter ions, or a fraction of a polyvalent counter ion sufficient to balance the cationic charge in the repeating unit which forms the ionene polymer backbone. Preferably, X²⁻ is two monovalent anions selected from a halide anion and a trihalide anion and more preferably, chloride or bromide. Ionene polymers having trihalide counter ions are described, for example, in U.S. Pat. No. 3,778,476, the disclosure of which is incorporated herein in its entirety by reference.

Ionene polymers having the repeating unit of formula (I) may be prepared by any of the methods known to one of ordinary skill in the art. One such method is to react a diamine of the formula R¹R²N—B—NR³R⁴ with a dihalide of the formula X—A—X, wherein the substituents R¹, R², R³, R⁴, A, X, and B have the same meanings as in formula (I). Ionene polymers having this repeating unit and methods for their preparation are described, for example, in U.S. Pat. Nos. 3,874,870; 3,931,319; 4,025,627; 4,027,020; 4,506,081; and 5,093,078, the disclosures of all of which are incorporated herein in their entireties by reference. The biological activity of ionene polymers having the repeating unit of formula (I) is also described in these patents.

A second type of ionene polymer that can be used in the present invention comprises the repeating unit of formula (II):

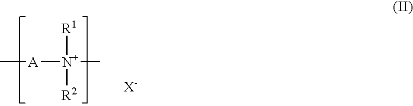

wherein R¹, R², and A are as defined above for formula (I). X⁻ is a monovalent counter ion, one-half of a divalent counter ion, or a fraction of a polyvalent counter ion sufficient to balance the cationic charge of the repeating unit which forms the ionene polymer. X⁻ can be, for example, a halide or trihalide anion, and is preferably chloride or bromide.

Ionene polymers having the repeating unit of formula (II) may also be prepared by any of the known methods. One method is to react an amine of the formula R¹R²N with a haloepoxide such as epichlorohydrin or (chloromethyl) oxirane, wherein R¹ and R² have the same meanings as in formula (I). Ionene polymers having the repeating unit of formula (II) are, for example, described in U.S. Pat. Nos. 4,111,679 and 5.051,124, the disclosures of which are incorporated herein in their entireties by reference. The biological activity of ionene polymers having the repeating unit of formula (II) is also described in these patents.

A third type of ionene polymer that can be used in the present invention comprises a repeating unit of formula (III):

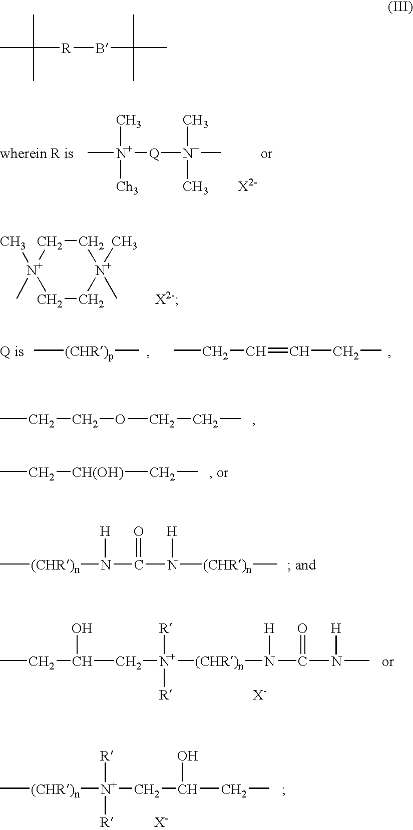

wherein n and p are integers and independently vary from 2 to 12; each R′ is independently hydrogen or a lower alkyl group (e.g., C₁-C₁₂ alkyl) wherein the alkyl group is unsubstituted or substituted; X²⁻ is a divalent counter ion, two monovalent counter ions, or a fraction of a polyvalent counter ion sufficient to balance the cationic charge in the group R; and X⁻ is a monovalent counter ion, one-half of a divalent counter ion, or a fraction of a polyvalent counter ion sufficient to balance the cationic charge in the group B′.

Preferably, R′ is hydrogen or a C₁-C₄ alkyl; n is 2-6 and p is 2-6. Most preferably, R′ is hydrogen or methyl, n is 3 and p is 2. Preferred counter ions, X²⁻ and X⁻ are the same as those discussed above with respect to formulae (I) and (II).

The polymers of formula (III) may be derived from bis(dialkylaminoalkyl) ureas, which are also known as urea diamines, by known methods. Ionene polymers of formula (III), methods of their preparation, and their biological activities are, for example, described in U.S. Pat. No. 4,506,081; the disclosure of which is incorporated herein in its entirety by reference.

Ionene polymers comprising the repeating units of formulae (I), (II), and (III) may also be cross-linked with primary, secondary, or other polyfunctional amines using means known in the art. Ionene polymers can be cross-linked either through the quaternary nitrogen atom, or through another functional group attached to the polymer backbone or to a side chain.

Cross-linked ionene polymers, prepared using cross-linking coreactants, are disclosed in U.S. Pat. No. 3,738,945 and U.S. Pat. No. RE 28,808, the disclosures of each of which are incorporated herein in their entireties by reference. U.S. Pat. No. RE 28,808 describes the cross-linking of ionene polymers prepared by the reaction of dimethylamine and epichlorohydrin. The cross-linking co-reactants listed are ammonia, primary amines, alkylenediamines, polyglycolamines, piperazines, heteroaromatic diamines, and aromatic diamines.

U.S. Pat. No. 5,051,124, the disclosure of which is incorporated herein in its entirety by reference, describes cross-linked ionene polymers resulting from the reaction of dimethylamine, a polyfunctional amine, and epichlorohydrin. Methods of inhibiting the growth of microorganisms using such cross-linked ionene polymers are also described.

Other examples of various cross-linked ionene polymers and their properties are provided in U.S. Pat. Nos. 3,894,946; 3,894,947; 3,930,877; 4,104,161; 4,164,521; 4,147,627; 4,166,041; 4,606,773; and 4,769,155. The disclosures of each of these patents are incorporated herein in their entireties by reference.

The ionene polymers comprising the repeating units of formulae (I), (II), or (III) may also be capped, i.e., have a specific end group. Capping may be achieved by any means known in the art. For example, an excess of one of the reactants used to make the ionene polymer can be employed to provide a capping group. Alternatively, a calculated quantity of a monofunctional tertiary amine or monofunctional substituted or unsubstituted alkyl halide can be reacted with an ionene polymer to obtain a capped ionene polymer. Ionene polymers can be capped at one or both ends. Capped ionene polymers and their microbicidal properties are described in U.S. Pat. Nos. 3,931,319 and 5,093,078. The disclosures of each of these patents are incorporated herein in their entireties by reference.

The specific ionene polymer employed is preferably selected based on the compatibility with the medium of intended use. Compatibility is determined by criteria such as solubility in the fluid system and lack of reactivity with the fluid or material or media in question. The compatibility is readily determined by one of ordinary skill by adding the ionene polymer to the material or media to be used. When used in a fluid system, it is preferable that the ionene polymer be freely soluble in the particular fluid, resulting in a uniform solution or dispersion.

Among the ionene polymers discussed above, a particularly preferred ionene polymer having a repeating unit of formula (I) is poly(oxyethylene(dimethyliminio)ethylene(di-methyliminio)ethylene dichloride). In this ionene polymer, R¹, R², R³, and R⁴ are each methyl, A is —CH₂CH₂OCH₂CH₂—, B is —CH₂CH₂—, and X²⁻ is 2Cl⁻, and the average molecular weight is 1,000-5,000. This ionene polymer is available from Buckman Laboratories, Inc. of Memphis, Tenn. as Busan® 77 product, a 60% aqueous dispersion of the polymer, or WSCP® product, a 60% aqueous dispersion of the polymer. Busan® 77 and WSCP® products are biocides used primarily in aqueous systems, including metalworking fluids for microorganism control.

Another particularly preferred ionene polymer having a repeating unit of formula (I), also available from Buckman Laboratories, Inc. as Busan® 79 product, or WSCP II product is the ionene polymer where R¹, R², R³, and R⁴ are each methyl, A is —CH₂CH(OH)CH₂—, B is —CH₂CH₂—, and X²⁻ is 2Cl⁻. This ionene polymer is a reaction product of N,N,N′,N′-tetramethyl-1,2-ethanediamine, with (chloromethyl)oxirane, and has a 1,000-5,000 average molecular weight. The polymer product Busan® 79 or WSCP® II product is a 60% aqueous solution of the polymer.

Preferred ionene polymers having the repeating unit of formula (II) are those where R¹ and R² are each methyl, A is —CH₂CH(OH)CH₂—, and X⁻ is Cl⁻. Busan® 1055 product is a 50% aqueous dispersion of such an ionene polymer obtained as a reaction product of dimethylamine with (chloromethyl)oxirane having a 2,000-10,000 average molecular weight. Busan 1055 is also known as APCA.

Busan® 1157 product is a 50% aqueous dispersion of the ionene polymer having the repeating unit of formula (II), obtained as a reaction product of dimethylamine with epichlorohydrin, cross-linked with ethylenediamine, where R¹ and R² are each methyl, A is —CH₂CH(OH)CH₂— and X⁻ is Cl⁻. This ionene polymer has a 100,000-500,000 average molecular weight.

Busan® 1155 product is a 50% aqueous dispersion of an ionene polymer having the repeating unit of formula (II), where R¹ and R2 are each methyl, A is —CH₂CH(OH)CH₂—, X⁻ is Cl⁻ and the ionene polymer is cross-linked with ammonia. This ionene polymer has a molecular weight of approximately 100,000-500,000.

Busan® 1099 product or Bubond® 65 product is a 25% aqueous dispersion of a cross-linked ionene polymer having repeating units of formula (II), where R¹ and R² are each methyl, A is —CH₂CH(OH)CH₂—, X⁻ is Cl⁻, the cross-linking agent is monomethylamine. This ionene polymer has a molecular weight of approximately 10,000-100,000.

Preferred ionene polymers having the repeating unit of formula (III) are those where R is a urea diamine and X²⁻ is 2Cl⁻, B′ is CH₂CH(OH)CH₂, and X⁻ is Cl⁻. BL® 1090 is a 50% aqueous dispersion of the ionene polymer obtained as a reaction product of N,N′-bis-[1-(3-(dimethylamino))-propyl]urea and epichlorohydrin, such an ionene polymer having a 2,000-15,000, preferably 3,000-7,000, average molecular weight.

Each of the above ionene polymers and products identified by trade name is available from Buckman Laboratories, Inc. of Memphis, Tenn.

The preparation of triclosan is described in US Pat. Nos. 3,629,477 and 6,133,483, and both disclosures are incorporated in their entirety herein by reference. Triclosan is commercially available and also easily synthesized from commercially available raw materials. A preferred halogenated diphenylether is 2,4,4′-tricloro-2′-hydroxydiphenyl ether, or triclosan.

As described above, components (a) an ionene polymer and (b) triclosan are used in a synergistically effective amounts. The weight ratios of (a) to (b) vary depending on the type of microorganisms and product, material, or media to which the composition is applied. In view of the present invention, one skilled in the art can readily determine, without undue experimentation, the appropriate weight ratios for a specific application. The ratio of component (a) to component (b) preferably ranges from 1:99 to 99:1, more preferably from 1:30 to 30:1, and most preferably 1:2 to 2:1.

Depending upon the specific application, the composition can be prepared in liquid form by dissolving the composition in water or in an organic solvent, or in dry form by adsorbing onto a suitable vehicle, or compounding into a tablet form. The preservative containing the composition of the present invention may be prepared in an emulsion form by emulsifying it in water, or if necessary, by adding a surfactant. Additional chemicals, such as insecticides, may be added to the foregoing preparations depending upon the intended use of the preparation.

The mode as well as the rates of application of the composition of this invention could vary depending upon the intended use. The composition could be applied by spraying or brushing onto the material or product. The material or product in question could also be treated by dipping in a suitable formulation of the composition. In a liquid or liquid-like medium, the composition could be added into the medium by pouring, or by metering with a suitable device so that a solution or a dispersion of the composition can be produced.

The synergistic activity of the combinations described above has been confirmed using standard laboratory techniques as illustrated below. The following examples are intended to illustrate, not limit, the present invention.

Microbicidal Evaluation

A. Fungal Evaluation

Mineral salts-glucose medium was used. To prepare the medium, the following ingredients were added to 1 liter of deionized water: 0.7 g of KH₂PO₄; 0.7 g of MgSO₄.7H₂O; 1.0 g of HN₄NO₃; 0.005 g of NaCl; 0.002 g of FeSO₄.7H₂0; 0.002 g ZnSO₄.7H₂O; 0.001 g of MnSO₄.7H₂O; 10 g of Glucose. The pH of the medium was adjusted to 6 with 1N NaOH. The medium was distributed in 5 ml amounts in test tubes and autoclaved at 121° C. for 20 minutes. The fungus, Trichoderma viride or Aspergillus niger, was grown on potato dextrose agar slant for 5 to 10 days and a spore suspension prepared by washing down the spores from the slant into a sterile saline solution. After addition of the biocides in the desired concentrations in the sterile mineral salts-glucose medium, the fungal spore suspension was added. The final spore concentration was approximately 10⁶ cfu/ml. The inoculated media was incubated at 25° C. for 7 days.

B. Bacterial Evaluation

Nutrient broth (2.5 g/liter of deionized water) was prepared. This was distributed in 5 ml amounts in test tubes and autoclaved at 121° C. for 20 minutes. After addition of the biocides in the desired concentrations to the nutrient broth, 100 microliters of a 24-hour grown suspension of Pseudomonas aeruginosa or Klebsiella pneumoniae cells of approximately 9.3×10⁸ cfu/ml were added and incubated at 30° C. for 24 or 48 hours.

C. Algal Evaluation

The medium used for testing the effectiveness of the invention against algae was modified Allen's medium (see Allen, A. A., Simple conditions for growth of unicellular blue-green algae on plates, JOURNAL OF PHYCOLOGY. 4:1-4 (1986)). To make modified Allen's medium, the following six individual stock solutions in g/200 ml of deionized water were first prepared: 1.50 g of K₂HPO₄; 1.50 g of MgSO₄.7H₂0; 0.80 g Na₂CO₃; 0.5 g CaCl₂.2H₂O; 1.16 g Na₂SiO₃.9H₂O; 1.20 g citric acid. A micronutrient stock solution was also prepared by adding the following ingredients to 1 liter of deionized water: 0.75 g of Na₂EDTA; 0.097 g FeCl₃.6H₂O; 0.041 g of MnCl₂.4H₂O; 0.005 g ZnCl₂; 0.002 g CoCl₂.6H₂O; 0.004 g Na₂MoO₄.2H₂O. To make the final modified Allen's medium, the following ingredients and the stock solutions, prepared from above, were combined in a flask and then made up to 1 liter with deionized water: 1.5 g NaNO₃; 5 ml K₂HPO₄; 5 ml MgSO₄.7H₂O; 5 ml Na₂CO₃; 10 ml CaCl₂.2H₂O; 10 ml Na₂SiO₃.9H₂O; 1 ml citric acid; and 1 ml micronutrient stock solution. The pH was adjusted to 7.8. Then the medium was dispensed in 5 ml amounts in test tubes and autoclaved at 121° C. for 20 minutes. After autoclaving, the biocides were added to the medium in the desired concentrations. Then one milliliter of a two-week old culture of Chlorella sp. was added and incubated at a temperature of 25° C., and lighting of 180 ft-candle intensity for 3 or 4 weeks.

In examples 1-6, synergism was demonstrated in separate experiments by testing combinations of the ionene polymer poly[oxyethylene(dimethyliminio)-ethylene(dimethyliminio)ethylene dichloride] (Busan® 77), poly[hydroxyethylene-(dimethyliminio)-2-hydroxypropylene(dimethyliminio)methylene dichloride] (Busan® 1055 or APCA), or a cross-linked terpolymer of dimethylamine, epichlorohydrin and ethylenediamine (Busan® 1157), (designated component A), and triclosan (designated component B) in a series of tests in varying ratios and a range of concentrations against the fungi Trichoderma viride, and Aspergillus niger, the bacteria Pseudomonas aeruginosa and Klebsiella pneumoniae, and the alga Chlorella sp. using the methods described above.

The lowest concentration of each mixture or compound which completely prevented growth of the fungi for seven days, the alga for three or four weeks, and the bacteria for 24 or 48 hours was taken as the end point for synergism calculations. End points for the various mixtures were then compared with the end points for the pure active ingredients alone in concomitantly prepared flasks or test tubes.

Synergism was demonstrated by the method described by Kull, E. C., et al., Applied Microbiology 9:538-541 (1961):

QA/Qa+QB/Qb

wherein

Qa=Concentration of compound A in parts per million, acting alone, which produced an end point.

Qb=Concentration of compound B in parts per million, acting alone, which produced an end point.

QA=Concentration of compound A in parts per million, in the mixture, which produced an end point.

QB=Concentration of compound B in parts per million, in the mixture, which produced an end point.

When the sum of QA/Qa and QB/Qb is greater than one, antagonism is indicated. When the sum is equal to one, additivity is indicated. When the sum is less than one, synergism exists.

This procedure for demonstrating synergism of the compositions of this invention is a widely used and acceptable procedure. More detailed information is provided in the article by Kull et al. Further information concerning this procedure is contained in U.S. Pat. No. 3,231,509, the disclosure of which is herein incorporated in its entirety by reference.

Based on the above criteria, a synergistic activity against bacteria, fungi, algae, and mixtures thereof is observed when an ionene polymer is combined with triclosan. Examples showing synergistic results can be found in Tables 1-6.

In general, an effective fungicidal, bactericidal, or algicidal response can be obtained when the synergistic combination is employed in concentrations ranging about 0.01 ppm to 1% (i.e., 10,000 ppm) of the ionene polymer, preferably 0.1 to 5,000 ppm, and most preferably 0.1 ppm to 1000 ppm; and from about 0.01 to 5,000 ppm of triclosan, preferably 0.1 to 3,000 ppm, and most preferably, 0.1 to 1,000 ppm.

TABLE 1
Combinations of WSCP ® and Triclosan against fungi

| | Quantities producing end points (ppm)* | | | | |
|---|---|---|---|---|---|
| Test organism | Oa | OA | Ob | OB | SI** |
| Trichoderma | >600 | — | — | — | — |
| viride | — | 600 | — | 10 | <1.4 |
| | — | 300 | — | 10 | 0.9 |
| | — | 150 | — | 10 | 0.65 |
| | — | 60 | — | 10 | 0.5 |
| | — | 30 | — | 10 | 0.45 |
| | — | 15 | — | 10 | 0.43 |
| | — | 6 | — | 10 | 0.41 |
| | — | 3 | — | 10 | 0.41 |
| | — | 1.5 | — | 10 | 0.40 |
| | — | 0.6 | — | 10 | 0.40 |
| | — | 600 | — | 5 | <1.2 |
| | — | 300 | — | 5 | 0.7 |
| | — | 150 | — | 5 | 0.45 |
| | — | 60 | — | 5 | 0.3 |
| | — | 30 | — | 5 | 0.25 |
| | — | 15 | — | 5 | 0.23 |
| | — | 6 | — | 5 | 0.21 |
| | — | 3 | — | 5 | 0.21 |
| | — | 1.5 | — | 5 | 0.2 |
| | — | 0.6 | — | 5 | 0.2 |
| | — | 600 | — | 2.5 | <1.1 |
| | — | 300 | — | 2.5 | 0.6 |
| | — | 150 | — | 2.5 | 0.35 |
| | — | 60 | — | 2.5 | 0.2 |
| | — | 30 | — | 2.5 | 0.15 |
| | — | — | 25 | — | — |
| Aspergillus | >600 | — | — | — | — |
| niger | — | 600 | — | 5 | <1.5 |
| | — | 300 | — | 5 | <1 |
| | — | 150 | — | 5 | 0.75 |
| | — | 60 | — | 5 | 0.6 |
| | — | 30 | — | 5 | 0.55 |
| | — | 15 | — | 5 | 0.53 |
| | — | — | 10 | — | — |

SI** = OA/Oa + OB/Ob
Component A = WSCP ®
Component B = Triclosan

TABLE 2
Combinations of WSCP ® and Triclosan against bacteria and alga

| | Quantities producing end points (ppm)* | | | | |
|---|---|---|---|---|---|
| Test organism | Oa | OA | Ob | OB | SI** |
| Pseudomonas | 15 | — | — | — | — |
| Aeruginosa | — | 6 | — | 500 | <1.4 |
| | — | 6 | — | 250 | 0.9 |
| | — | 6 | — | 100 | 0.6 |
| | — | 6 | — | 50 | 0.5 |
| | — | 6 | — | 25 | 0.45 |
| | — | — | >500 | — | — |
| Chlorella sp. | >2 | — | — | — | — |
| | — | 0.1 | — | 0.4 | 0.85 |
| | — | 0.2 | — | 0.4 | 0.9 |
| | — | 0.3 | — | 0.4 | 0.95 |
| | — | 0.4 | — | 0.2 | 0.6 |
| | — | 0.4 | — | 0.3 | 0.8 |
| | — | 0.4 | — | 0.4 | <1 |
| | — | 0.5 | — | 0.2 | 0.65 |
| | — | 0.5 | — | 0.3 | 0.85 |
| | — | 0.6 | — | 0.2 | 0.7 |
| | — | 0.6 | — | 0.3 | 0.9 |
| | — | 0.7 | — | 0.2 | 0.75 |
| | — | 0.7 | — | 0.3 | 0.95 |
| | — | 0.8 | — | 0.2 | 0.8 |
| | — | 0.8 | — | 0.3 | <1 |
| | — | 1.0 | — | 0.2 | 0.9 |
| | — | — | 0.5 | — | — |

SI** = OA/Oa + OB/Ob
Component A = WSCP ®
Component B = Triclosan

TABLE 3
Combinations of APCA and Triclosan against fungi

| | Quantities producing end points (ppm)* | | | | |
|---|---|---|---|---|---|
| Test organism | Oa | OA | Ob | OB | SI** |
| Trichoderma | >600 | — | — | — | — |
| viride | — | 600 | — | 10 | <1.4 |
| | — | 300 | — | 10 | 0.9 |
| | — | 150 | — | 10 | 0.65 |
| | — | 60 | — | 10 | 0.5 |
| | — | 30 | — | 10 | 0.45 |
| | — | 15 | — | 10 | 0.43 |
| | — | 6 | — | 10 | 0.41 |
| | — | 3 | — | 10 | 0.41 |
| | — | 1.5 | — | 10 | 0.40 |
| | — | 600 | — | 5 | <1.2 |
| | — | 300 | — | 5 | 0.7 |
| | — | 150 | — | 5 | 0.45 |
| | — | 60 | — | 5 | 0.3 |
| | — | 30 | — | 5 | 0.25 |
| | — | 15 | — | 5 | 0.23 |
| | — | 6 | — | 5 | 0.21 |
| | — | 3 | — | 5 | 0.21 |
| | — | 600 | — | 2.5 | <1.1 |
| | — | 300 | — | 2.5 | 0.6 |
| | — | 150 | — | 2.5 | 0.35 |
| | — | — | 25 | — | — |
| Aspergillus | >600 | — | — | — | — |
| niger | — | 600 | — | 10 | <1.5 |
| | — | 300 | — | 10 | 0.9 |
| | — | 150 | — | 10 | 0.65 |
| | — | 60 | — | 10 | 0.5 |
| | — | 30 | — | 10 | 0.45 |
| | — | 15 | — | 10 | 0.43 |
| | — | 6 | — | 10 | 0.41 |
| | — | 1.5 | — | 10 | 0.40 |
| | — | 0.6 | — | 10 | 0.42 |
| | — | 600 | — | 5 | <1.2 |
| | — | 300 | — | 5 | 0.7 |
| | — | 150 | — | 5 | 0.45 |
| | — | 60 | — | 5 | 0.3 |
| | — | 30 | — | 5 | 0.25 |
| | — | 15 | — | 5 | 0.23 |
| | — | 6 | — | 5 | 0.21 |
| | — | 600 | — | 2.5 | <1.1 |
| | — | 300 | — | 2.5 | 0.6 |
| | — | 150 | — | 2.5 | 0.35 |
| | — | 60 | — | 2.5 | 0.2 |
| | — | 30 | — | 2.5 | 0.15 |
| | — | 15 | — | 2.5 | 0.13 |
| | — | — | 25 | — | — |

SI** = OA/Oa + OB/Ob
Note:
All concentrations reported are on active ingredient basis.
Component A = APCA
Component B = Triclosan

TABLE 4
Combinations of APCA anal Triclosan against bacteria and alga

| | Quantities producing end points (ppm)* | | | | |
|---|---|---|---|---|---|
| Test organism | Oa | OA | Ob | OB | SI** |
| Pseudomonas | 15 | — | — | — | — |
| aeruginosa | — | 6 | — | 500 | <1.4 |
| | — | 6 | — | 250 | 0.9 |
| | — | 6 | — | 100 | 0.6 |
| | — | 6 | — | 50 | 0.5 |
| | — | 6 | — | 25 | 0.45 |
| | — | 6 | — | 10 | 0.42 |
| | — | 6 | — | 5 | 0.41 |
| | — | 6 | — | 2.5 | 0.41 |
| | — | 6 | — | 1 | 0.40 |
| | — | — | >500 | — | — |
| Klebsiella | 15 | — | — | — | — |
| pneumoniae | — | 6 | — | 0.25 | 0.9 |
| | — | 3 | — | 0.25 | 0.7 |
| | — | 1.5 | — | 0.25 | 0.6 |
| | — | 6 | — | 0.10 | 0.6 |
| | — | 3 | — | 0.10 | 0.4 |
| | — | 1.5 | — | 0.10 | 0.3 |
| | — | — | 0.5 | — | — |
| Chlorella sp. | 1 | — | — | — | — |
| | — | 0.8 | — | 0.1 | 0.97 |
| | — | 0.5 | — | 0.2 | 0.83 |
| | — | 0.6 | — | 0.2 | 0.93 |
| | — | 0.4 | — | 0.3 | 0.9 |
| | — | 0.2 | — | 0.4 | 0.87 |
| | — | 0.3 | — | 0.4 | 0.97 |
| | — | 0.1 | — | 0.5 | 0.93 |
| | — | 0.6 | — | 0.2 | 0.93 |
| | — | — | 0.6 | — | — |

SI** = OA/Oa + OB/Ob
Component A = APCA
Component B = Triclosan

TABLE 5
Combinations of Busan ® 1157 and Triclosan against fungi

| | Quantities producing end points (ppm)* | | | | |
|---|---|---|---|---|---|
| Test organism | Oa | OA | Ob | OB | SI** |
| Trichoderma | >500 | — | — | — | — |
| viride | — | 500 | — | 10 | <1.4 |
| | — | 250 | — | 10 | 0.9 |
| | — | 125 | — | 10 | 0.65 |
| | — | 50 | — | 10 | 0.5 |
| | — | 25 | — | 10 | 0.45 |
| | — | 12.5 | — | 10 | 0.43 |
| | — | 5 | — | 10 | 0.41 |
| | — | 2.5 | — | 10 | 0.41 |
| | — | 1.23 | — | 10 | 0.40 |
| | — | 0.5 | — | 10 | 0.40 |
| | — | 500 | — | 5 | <1.2 |
| | — | 250 | — | 5 | 0.7 |
| | — | 125 | — | 5 | 0.45 |
| | — | 50 | — | 5 | 0.3 |
| | — | 25 | — | 5 | 0.25 |
| | — | — | 25 | — | — |
| Aspergillus | >500 | — | — | — | — |
| Niger | — | 500 | — | 5 | <1.5 |
| | — | 250 | — | 5 | 1 |
| | — | 125 | — | 5 | 0.75 |
| | — | 50 | — | 5 | 0.6 |
| | — | 25 | — | 5 | 0.55 |
| | — | 12.5 | — | 5 | 0.53 |
| | — | 5 | — | 5 | 0.51 |
| | — | 2.5 | — | 5 | 0.51 |
| | — | — | 10 | — | — |

SI** = OA/Oa + OB/Ob
Component A = Busan ® 1157
Component B = Triclosan

TABLE 6
Combinations of Busan ® 1157 and Triclosan
against two bacteria and alga

| | Quantities producing end points (ppm)* | | | | |
|---|---|---|---|---|---|
| Test organism | Oa | OA | Ob | OB | SI** |
| Pseudomonas | 12.5 | — | — | — | — |
| Aeruginosa | — | 5 | — | 500 | <1.4 |
| | — | 5 | — | 250 | 0.9 |
| | — | 5 | — | 100 | 0.6 |
| | — | 5 | — | 50 | 0.5 |
| | — | 5 | — | 25 | 0.45 |
| | — | 5 | — | 10 | 0.42 |
| | — | 5 | — | 5 | 0.41 |
| | — | 5 | — | 2.5 | 0.41 |
| | — | 5 | — | 1 | 0.40 |
| | — | — | >500 | — | — |
| Klebsiella | 12.5 | — | — | — | — |
| Pneumoniae | — | 5 | — | 0.25 | 0.9 |
| | — | 2.5 | — | 0.25 | 0.7 |
| | — | 5 | — | 0.10 | 0.6 |
| | — | 2.5 | — | 0.10 | 0.4 |
| | — | — | 0.5 | — | — |
| Chlorella sp. | 1 | — | — | — | — |
| | — | 0.1 | — | 0.5 | 0.93 |
| | — | 0.6 | — | 0.2 | 0.93 |
| | — | — | 0.6 | — | — |

SI** = OA/Oa + OB/Ob
Component A = Busan ® 1157
Component B - Triclosan

As shown in the above examples, the present invention is quite effective as a antimicrobial agent, and preferably provides synergistic results. One of the important aspects of the present invention is the ability to have effectiveness against Pseudomonas sp., especially with relatively low concentrations of triclosan and ionene polymers. It is interesting to note that when triclosan and ionene polymers are used individually, triclosan is ineffective against Pseudomonas sp., and relatively high concentration of ionene polymers are required to control this organism. This is an additional benefit and unexpected benefit of the present invention.

Other embodiments of the present invention, will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A composition comprising (a) an ionene polymer and (b) a halogenated diphenyl ether, wherein components (a) and (b) are present in a synergistically microbicidally effective combined amount to control the growth of at least one microorganism.

2. The composition of claim 1, wherein the ionene polymer comprises the repeating unit of formula (I): 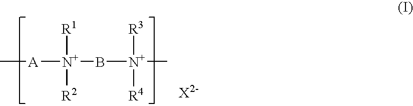
wherein R¹, R², R³, and R⁴ are identical or different, and are selected from hydrogen, C₁-C₂₀ alkyl optionally substituted with at least one hydroxyl group, or benzyl optionally substituted on the benzene moiety with at least one C₁-C₂₀, alkyl group;
A is a divalent radical selected from C₁-C₁₀ alkyl, C₂-C₁₀ alkenyl, C₂-C₁₀ alkynyl, C₁-C₁₀ hydroxyalkyl, symmetric or asymmetric di-C₁-C₁₀-alkylether, aryl, aryl-C₁-C₁₀-alkyl, or C₁-C₁₀-alkylaryl-C₁-C₁₀-alkyl;
B is a divalent radical selected from C₁-C₁₀ alkyl, C₂-C₁₀ alkenyl, C₂-C₁₀ alkynyl, C₁-C₁₀ hydroxyalkyl, symmetric or asymmetric di-C₁-C₁₀-alkylether, aryl, aryl-C₁-C₁₀-alkyl, or C₁-C₁₀-alkylaryl-C₁-C₁₀-alkyl; and
X²⁻ is a divalent counter ion, two monovalent counter ions, or a fraction of a polyvalent counter ion sufficient to balance the cationic charge in the repeating unit which forms the ionene polymer backbone.

3. The composition of claim 1, wherein the microorganism is bacteria, fungi, algae, or mixtures thereof.

4. The composition of claim 2, wherein the C₁-C₂₀ alkyl is substituted with at least one hydroxyl group.

5. The composition of claim 2, wherein benzyl is substituted on the benzene moiety with at least one C₁-C₂₀ alkyl group.

6. The composition of claim 1, wherein the ionene polymer is capped or cross-linked.

7. The composition of claim 1, wherein the halogenated diphenyl ether is 2,4,4′-trichloro-2′-hydroxydiphenylether.

8. The composition of claim 1, wherein the halogenated diphenyl ether is triclosan.

9. The composition of claim 1, wherein the weight ratio of (a) to (b) is from about 1:99 to about 99:1.

10. The composition of claim 8, wherein the weight ratio of (a) to (b) is from about 1.30 to about 30:1.

11. The composition of claim 9, wherein the weight ratio of (a) to (b) is from about 1:2 to about 2:1.

12. The composition of claim 2, wherein R¹, R², R³, and R⁴ are each methyl, A is —CH₂CH₂OCH₂CH₂—, B is —CH₂CH₂—, and X²⁻ is 2Cl⁻.

13. The composition of claim 2, wherein R¹, R², R³, and R⁴ are each methyl, A is —CH₂CH(OH)CH₂—, B is —CH₂CH₂—, and X²⁻ is 2Cl⁻.

14. The composition of claim 2, wherein R¹, R², R³, and R⁴ are different.

15. The composition of claim 2, wherein R¹, R², R³, and R⁴ are the same.

16. The composition of claim 2, wherein A is a C₁-C₂₀ alkyl and B is a C₁-C₁₀ alkyl.

17. The composition of claim 2, wherein A is a C₂-C₁₀ alkenyl.

18. The composition of claim 2, wherein B is a C₂-C₁₀ alkenyl.

19. The composition of claim 2, wherein A is a C₂-C₁₀ alkynyl and B is a C₂-C₁₀ alkynyl.

20. A method of controlling the growth of at least one microorganism in or on a product, material, or medium susceptible to attack by a microorganism, the method comprising adding to the product, material, or medium the composition of claim 1.

21. A method of controlling the growth of at least one microorganism in or on a product, material, or medium susceptible to attack by a microorganism, the method comprising adding to the product, material, or medium the composition of claim 2.

22. The method of claim 20, wherein the microorganism is bacteria, fungi, algae, or mixtures thereof.

23. The method of claim 20, wherein the material or medium is wood pulp, wood chips, lumber, paints, leathers, adhesives, coatings, animal hides, tanning liquor, paper mill liquor, metalworking fluids, petrochemicals, pharmaceutical formulations, cooling water, recreational water, dyes, clays, mineral slurries, cationic surfactants, formulations with cationic surfactants, influent water, waste water, pasteurizers, retort cookers, cosmetic formulations, toiletry formulations, textiles, geological, drilling lubricants, or agrochemical compositions for crop or seed protection.

24. The method of claim 20, wherein the material or medium is in the form of a solid, a dispersion, an emulsion, or a solution.

25. The composition of claim 21, wherein the C₁-C₂₀ alkyl is substituted with at least one hydroxyl group.

26. The method of claim 21, wherein benzyl is substituted on the benzene moiety with at least one C₁-C₂₀ alkyl group.

27. The method of claim 20, wherein the halogenated diphenyl ether is 2,4,4′-trichloro-2′-hydroxydiphenylether.

28. The method of claim 20, wherein the halogenated diphenyl ether is triclosan.

29. The method of claim 20, wherein the weight ratio of (a) to (b) is from about 1:99 to about 99:1.

30. The method of claim 20, wherein the weight ratio of (a) to (b) is from about 1:30 to about 30:1.

31. The method of claim 20, wherein the weight ratio of (a) to (b) is from about 1:2 to about 2:1.

32. The method of claim 21, wherein R¹, R², R³, and R⁴ are each methyl, A is —CH₂CH₂ CH₂CH₂—, B is —CH₂CH₂—, and X²⁻ is 2Cl⁻.

33. The method of claim 21, wherein R¹, R², R³, and R⁴ are each methyl, A is —CH₂CH(OH)CH₂—, B is —CH₂CH₂—, and X²⁻ is 2Cl⁻.

34. The method of claim 21, wherein R¹, R², R³, and R⁴ are different.

35. The method of claim 21, wherein R¹, R², R³, and R⁴ are the same.

36. The method of claim 21, wherein A is a C₁-C₂₀ alkyl and B is a C₁-C₁₀ alkyl.

37. The method of claim 21, wherein A is a C₂-C₁₀ alkynyl and B is a C₂-C₁₀ alkynyl.

38. A method for preventing spoilage of a product, material, or medium caused by microorganism selected from bacteria, fungi, algae, or mixtures thereof, wherein the method comprises adding to the product, material, or medium a composition of claim 1.

39. The method of claim 38, wherein the material is seeds or crops.

40. The method of claim 38, wherein the halogenated diphenyl ether is 2,4,4′-trichloro-2′-hydroxydiphenylether.

41. The method of claim 38, wherein the halogenated diphenyl ether is triclosan.

42. The composition of claim 1, wherein said ionene polymer comprises the repeating unit of formula (II): 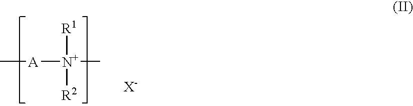
wherein R¹, and R² are identical or different, and are selected from hydrogen, C₁-C₂₀ alkyl optionally substituted with at least one hydroxyl group, or benzyl optionally substituted on the benzene moiety with at least one C₁-C₂₀, alkyl group;
A is a divalent radical selected from C₁-C₁₀ alkyl, C₂-C₁₀ alkenyl, C₂-C₁₀ alkynyl, C₁-C₁₀ hydroxyalkyl, symmetric or asymmetric di-C₁-C₁₀-alkylether, aryl, aryl-C₁-C₁₀-alkyl, or C₁-C₁₀-alkylaryl-C₁-C₁₀-alkyl; and X⁻ is a monovalent counter ion, one-half of a divalent counter ion, or a fraction of a polyvalent counter ion sufficient to balance the cationic charge of the repeating unit which forms the ionene polymer.

43. The composition of claim 1, wherein said ionene polymer comprises a repeating unit of formula (III): 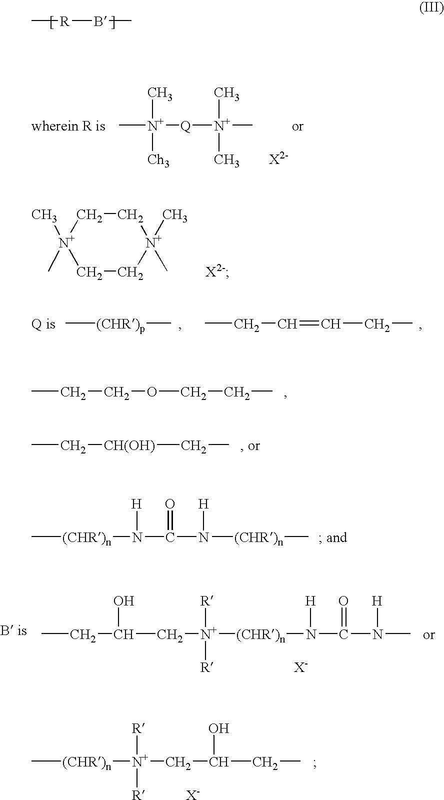
wherein n and p are integers and independently vary from 2 to 12; each R′ is independently hydrogen or a lower alkyl group (e.g., C₁-C₁₂ alkyl) wherein the alkyl group is unsubstituted or substituted; X²⁻ is a divalent counter ion, two monovalent counter ions, or a fraction of a polyvalent counter ion sufficient to balance the cationic charge in the group R; and X⁻ is a monovalent counter ion, one-half of a divalent counter ion, or a fraction of a polyvalent counter ion sufficient to balance the cationic charge in the group B′.

44. The composition of claim 43, wherein R′ is hydrogen or a C₁-C₄ alkyl, n is 2-6, and p is 2-6.

45. The composition of claim 43, wherein R′ is hydrogen or methyl, n is 3, and p is 2.

46. A method of controlling the growth of at least one microorganism in or on a product, material, or medium susceptible to attack by a microorganism, the method comprising adding to the product, material, or medium the composition of claim 42.

47. A method of controlling the growth of at least one microorganism in or on a product, material, or medium susceptible to attack by a microorganism, the method comprising adding to the product, material, or medium the composition of claim 43.

48. A method for preventing spoilage of a product, material, or medium caused by microorganism selected from bacteria, fungi, algae, or mixtures thereof, wherein the method comprises adding to the product, material, or medium a composition of claim 2.

49. A method for preventing spoilage of a product, material, or medium caused by microorganism selected from bacteria, fungi, algae, or mixtures thereof, wherein the method comprises adding to the product, material, or medium a composition of claim 42.

50. A method for preventing spoilage of a product, material, or medium caused by microorganism selected from bacteria, fungi, algae, or mixtures thereof, wherein the method comprises adding to the product, material, or medium a composition of claim 43.
